SEQUENCE LISTING (3 sequences)

SEQ ID NO: 1 - DNA, 20 bp, synthetic construct
  source 1..20: mol_type = other DNA
  misc_feature 1..20: note = forward primer
aaattttggggaccaggaac

SEQ ID NO: 2 - DNA, 20 bp, synthetic construct
  misc_feature 1..20: note = Reverse Primer
  source 1..20: mol_type = other DNA
tggcagctgtgtaggtcaac

SEQ ID NO: 3 - DNA, 20 bp, synthetic construct
  misc_feature 1..20: note = Probe
  source 1..20: mol_type = other DNA
  modified_base 1: mod_base = OTHER; note = carboxyfluorescein
  modified_base 20: mod_base = OTHER; note = black hole quencher 1
atgtcgcgcattggcatgga

(19) United States
(12) Patent Application Publication
(10) Pub. No.: US 2024/0110235 A1
(43) Pub. Date: Apr. 4, 2024

(54) REACTION COMPOSITION FOR NUCLEIC ACID AMPLIFICATION AND NUCLEIC ACID AMPLIFICATION METHOD USING SAME

(71) Applicant: NOF CORPORATION, Tokyo (JP)

(72) Inventors: Hirotaka SUZUKI, Kawasaki (JP); Masaru MATSUDA, Kawasaki (JP)

(73) Assignee: NOF CORPORATION, Tokyo (JP)

(21) Appl. No.: 18/458,354

(22) Filed: Aug. 30, 2023

(30) Foreign Application Priority Data
Oct. 3, 2022 (JP) 2022-159813

(51) Int. Cl. C12Q 1/686; C12Q 1/6848

(52) U.S. Cl. CPC C12Q 1/686; C12Q 1/6848

(57) ABSTRACT

A method for improving repeatability of a nucleic acid amplification method is provided by the present invention, the method including performing a nucleic acid amplification reaction using a reaction composition containing water, an amplification target nucleic acid at a concentration of not more than 10,000 copies/mL, and one or more polymers selected from the following group A at a concentration of 0.005 to 0.5 w/v %:

[group A]

(A1) a polymer consisting of a constitutional unit derived from 2-(meth)acryloyloxyethylphosphorylcholine

(A2) a copolymer containing a constitutional unit derived from 2-(meth)acryloyloxyethylphosphorylcholine and a constitutional unit derived from (meth)acrylic acid.

### BACKGROUND OF THE INVENTION

### Field of the Invention

The present invention relates to a reaction composition for nucleic acid amplification (specifically a reaction composition used to improve repeatability of a nucleic acid amplification method) and a nucleic acid amplification method using same.

### Discussion of the Background

A nucleic acid amplification method is a method for amplifying a target nucleic acid from several copies to tens of thousands of copies, and is actively used in various fields for gene cloning and clinical test. Particularly, in nucleic acid amplification tests, a nucleic acid amplification method is used to determine the presence or absence of microorganisms (e.g., bacteria, archaebacteria, and fungi), viruses, cells, genes, and the like, as well as the amounts thereof present. The nucleic acid amplification tests are used in a variety of fields, including medical field, veterinary medicine, environmentology, food and pharmaceutical manufacturing, molecular biology research, forensic medicine, and so forth. Among them, tests aiming at determining the presence or absence of target nucleic acids can be called nucleic acid amplification qualitative tests.

A representative method of nucleic acid amplification method is the Polymerase Chain Reaction (PCR) method. In a typical PCR method, a region of interest in a nucleic acid is replicated and amplified two-fold per cycle under ideal conditions by repeating a cycle consisting of the three steps of (1) denaturing template DNA (separation from double-stranded DNA to single-stranded DNA), (2) annealing of primer to single-stranded template DNA, and (3) elongating primer by DNA polymerase.

A typical PCR method is a method for amplifying DNA by using the DNA as a template. On the other hand, a reverse transcription polymerase chain reaction method (hereinafter sometimes to be abbreviated as “RT-PCR method”) uses RNA as a template to amplify DNA. That is, a complementary DNA (hereinafter sometimes to be abbreviated as “cDNA”) is synthesized from template RNA by a reverse transcription reaction, and the resulting cDNA is used as a template to further perform PCR. The RT-PCR method is further divided into a 2-step method in which reverse transcription reaction and qPCR method are performed in separate containers, and a 1-step method in which these are performed as a series of reactions in the same container.

Furthermore, various isothermal amplification methods have been developed, such as the LAMP (Loop-mediated isothermal amplification) method, which combines strand displacement reactions to amplify nucleic acids under isothermal conditions without thermal cycling, and the NEAR method (Nicking endonuclease amplification reaction) method, which performs nucleic acid amplification by combining nicking enzymes under isothermal conditions.

Among the aforementioned nucleic acid amplification methods, the type of nucleic acid amplification method that considers the presence or absence and amount of the final amplification product is particularly called the end-point method, and can be used for nucleic acid amplification qualitative tests to determine the presence or absence of target nucleic acids.

A nucleic acid amplification method of the type that monitors the progress of the nucleic acid amplification reaction by measuring fluorescence and turbidity during the nucleic acid amplification reaction, and quantifies the target nucleic acid based on the speed of a signal to become detectable is called a real-time method. A real-time method using only the presence or absence of detection as a criterion can also be used for nucleic acid amplification qualitative tests.

In recent years, moreover, a method called a digital PCR method (hereinafter sometimes to be abbreviated as “dPCR method”) has also been developed in which nucleic acid amplification reaction liquids are distributed into many microcompartments to decrease the concentration of the target nucleic acid per compartment extremely low, nucleic acid amplification is performed simultaneously for all compartments in this state, and the target nucleic acid is quantified using a statistical model from the proportion of the compartments with amplification. The reaction performed within each compartment of dPCR is end-point nucleic acid amplification.

### SUMMARY OF THE INVENTION

Low repeatability often poses a problem in the aforementioned nucleic acid amplification qualitative tests described above. In particular, it is known that simultaneous repeatability decreases under conditions where a sample contains a target nucleic acid to be amplified at an extremely low concentration, that is, the probability of correctly determining a sample containing a target nucleic acid as positive decreases. Therefore, various techniques have been devised to improve the repeatability of nucleic acid amplification tests. For example, a so-called “hot start method” for nucleic acid amplification reaction is well known, in which an anti-DNA polymerase antibody or the like is used in combination in a nucleic acid amplification reaction, and DNA polymerase is encapsulated at around room temperature. However, the repeatability of the nucleic acid amplification qualitative test is currently insufficient even when this method is applied.

In addition, JP-A-2005-000162, which is incorporated herein by reference in its entirety, and JP-A-2007-195487, which is incorporated herein by reference in its entirety, disclose methods for improving the repeatability of nucleic acid amplification tests by devising the sequence design of primers to be used in nucleic acid amplification reactions. However, since these methods require changes of primer designs, they are insufficient in applicability to various test subjects.

WO 2022/163506, which is incorporated herein by reference in its entirety, discloses addition of a polymer having a phosphorylcholine group to a reaction composition for nucleic acid amplification. However, in WO 2022/163506, detection sensitivity of nucleic acids is examined, and repeatability of nucleic acid amplification methods (particularly, repeatability of a nucleic acid amplification method for an amplification target nucleic acid with an extremely low concentration) is not examined.

The present invention aims to provide a reaction composition for nucleic acid amplification that can improve repeatability of a nucleic acid amplification method (particularly, a nucleic acid amplification method for an amplification target nucleic acid with an extremely low concentration).

The present inventors have conducted intensive studies in an attempt to achieve the aforementioned goal and found that repeatability of a nucleic acid amplification method for an amplification target nucleic acid with an extremely low concentration can be improved by using one or more polymers selected from the following group A. The present invention based on this finding provides the following.
[1] A reaction composition for use in improving repeatability of a nucleic acid amplification method, comprising water, an amplification target nucleic acid at a concentration of not more than 10,000 copies/mL, and one or more polymers selected from the following group A at a concentration of 0.005 to 0.5 W/V %:
[group A]
(A1) a polymer consisting of a constitutional unit derived from 2-(meth)acryloyloxyethylphosphorylcholine
(A2) a copolymer containing a constitutional unit derived from 2-(meth)acryloyloxyethylphosphorylcholine and a constitutional unit derived from (meth)acrylic acid.
[2] A nucleic acid amplification method using the reaction composition described in the aforementioned [1].
[3] A genetic testing method using the nucleic acid amplification method described in the aforementioned [2].
[4] A method for improving repeatability of a nucleic acid amplification method, comprising performing a nucleic acid amplification reaction using a reaction composition comprising water, an amplification target nucleic acid at a concentration of not more than 10,000 copies/mL, and one or more polymers selected from the following group A at a concentration of 0.005 to 0.5 w/v %:
[group A]
(A1) a polymer consisting of a constitutional unit derived from 2-(meth)acryloyloxyethylphosphorylcholine
(A2) a copolymer containing a constitutional unit derived from 2-(meth)acryloyloxyethylphosphorylcholine and a constitutional unit derived from (meth)acrylic acid.
[5] A genetic testing method using the method described in the aforementioned [4].

### Effects of the Invention

Using the reaction composition for nucleic acid amplification of the present invention, high repeatability is achieved in a nucleic acid amplification method using an amplification target nucleic acid at an extremely low concentration. Therefore, using the reaction composition for nucleic acid amplification of the present invention, high repeatability can be achieved in, for example, a nucleic acid amplification qualitative test using an amplification target nucleic acid with an extremely low concentration, and a positive judgment can be made with high precision. In addition, using the reaction composition for nucleic acid amplification of the present invention in, for example, an end-point nucleic acid amplification method performed in each compartment of dPCR, high repeatability can be achieved, and nucleic acids can be quantified with high precision by dPCR.

### DETAILED DESCRIPTION OF THE PREFERRED EMBODIMENTS

The present invention is described in detail below. When stepwise numerical ranges are described in the present specification, the lower limit and the upper limit of each numerical range can be combined. In the case of, for example, “preferably 10 to 100, more preferably 20 to 90”, the preferred lower limit 10 can be combined with the more preferred upper limit 90 (i.e., the numerical range of “10 to 90” is also within the range of the present specification). In the present specification, the “(meth)acrylic acid” basically means an “acrylic acid or methacrylic acid”. When a plurality of the (meth)acrylic acids may be present, the “(meth)acrylic acid” means an “acrylic acid and/or methacrylic acid”. Other terms similar to the “(meth)acrylic acid” also mean the same as the “(meth)acrylic acid”.

### [Reaction Composition]

The reaction composition for nucleic acid amplification of the present invention (sometimes to be abbreviated as “the reaction composition of the present invention” in the present specification) contains water, amplification target nucleic acid at a concentration of not more than 10,000 copies/mL, and one or more polymers selected from the following group A (sometimes to be abbreviated as “the polymer of the present invention” in the present specification) at a concentration of 0.005 to 0.5 w/v %.
[group A]
(A1) a polymer consisting of a constitutional unit derived from 2-(meth)acryloyloxyethylphosphorylcholine (hereinafter sometimes to be abbreviated as “polymer A1”)
(A2) a copolymer containing a constitutional unit derived from 2-(meth)acryloyloxyethylphosphorylcholine and a constitutional unit derived from (meth)acrylic acid (hereinafter sometimes to be abbreviated as “polymer A2”)

The reaction composition of the present invention is used to improve the repeatability of the nucleic acid amplification method. Examples of the nucleic acid amplification method include PCR method, LAMP method, NEAR method, TMA method (Transcription Mediated Amplification method), IICAN method (Isothermal and Chimeric primer-initiated Amplification of Nucleic acids method), SDA method (Strand Displacement Amplification method), LCR method (Ligase Chain Reaction method), NASBA method (Nucleic Acid Sequence-Based Amplification method), and the like. The nucleic acid amplification method is preferably a PCR method.

As the PCR method, an appropriate method may be selected according to the type of the nucleic acid to be amplified. When the amplification target nucleic acid is DNA, a typical PCR method is preferably selected, and when the amplification target nucleic acid is RNA, an RT-PCR method is preferably selected.

The reaction composition of the present invention contains an amplification target nucleic acid at a concentration of not more than 10,000 copies/mL. As used herein, the “concentration (copy/mL) of the nucleic acid in a reaction composition” means “the amount (copies) of nucleic acid per 1 mL of the reaction composition”.

As described later, (1) a nucleic acid amplification method using the reaction composition of the present invention (hereinafter sometimes to be abbreviated as “the nucleic acid amplification method of the present invention”) and (2) a method for improving repeatability of a nucleic acid amplification method, including performing a nucleic acid amplification reaction by using the reaction composition of the present invention (hereinafter sometimes to be abbreviated as “the improvement method of the present invention”) can be used in genetic testing methods. In the genetic testing method using the nucleic acid amplification method of the present invention or the improvement method of the present invention, the amplification target nucleic acid in the reaction composition of the present invention is a nucleic acid that may be contained in a sample for the genetic testing method. The sample for the genetic testing method may not contain the test target nucleic acid. Therefore, the concentration of an amplification target nucleic acid in the reaction composition of the present invention may be 0.

The concentration of an amplification target nucleic acid in the reaction composition of the present invention (concentration of nucleic acid before performing a nucleic acid amplification reaction) is, from the aspect of the effect of the present invention (repeatability), preferably not less than 50 copies/mL and not more than 10,000 copies/mL, more preferably not less than 100 copies/mL and not more than 5,000 copies/mL, further preferably not less than 250 copies/mL and not more than 2,000 copies/mL, particularly preferably not less than 500 copies/mL and not more than 1,000 copies/mL.

The reaction composition of the present invention contains the polymer of the present invention at a concentration of 0.005 to 0.5 w/v %. Here, the “concentration (w/v %) of a polymer in the reaction composition” means “the amount (g) of a polymer per 100 mL of the reaction composition”. When the reaction composition of the present invention contains two or more kinds of the polymer of the present invention, the aforementioned concentration means a total of the concentrations of the aforementioned polymers.

The concentration of the polymer of the present invention in the reaction composition of the present invention is preferably 0.01 to 0.1 w/v %, more preferably 0.05 to 0.1 w/v %, from the aspect of the repeatability.

### [Amplification Target Nucleic Acid]

The amplification target nucleic acid is DNA or RNA, and contains a sequence desired to be amplified by the nucleic acid amplification method.

Amplification target nucleic acids are provided, for example, as viruses, bacterial cells, cells, body fluids, tissues, and the like, suspensions thereof, nucleic acid extracts prepared therefrom, and the like. The viruses, bacterial cells, cells, bodily fluids, tissues, and the like may be those collected from humans, animals, or plants in the natural world or the environment, or may be those isolated and cultured. Amplification target nucleic acids may also be provided as in vitro synthetic products.

A known method can be used as a method for determining the nucleic acid concentration. For example, a method of calculating from the absorbance of the nucleic acid solution near 260 nm, a method of measuring fluorescence intensity by adding a fluorescent reagent such as ethidium bromide, SYBR™ Green, and the like, a method by gPCR method, a method by dPCR, and the like can be mentioned.

### [Polymer]

The polymers of the present invention (polymer A1 and polymer A2) are described below.

The “polymer consisting of a constitutional unit derived from 2-(meth)acryloyloxyethylphosphorylcholine” which is polymer A1 means a polymer whose constitutional units (repeating units) consist of constitutional units derived from 2-(meth)acryloyloxyethylphosphorylcholine. Polymer A1 may be a homopolymer of 2-(meth)acryloyloxyethylphosphorylcholine or a copolymer of 2-acryloyloxyethylphosphorylcholine and 2-methacryloyloxyethylphosphorylcholine.

When the polymer of the present invention is a copolymer, the copolymer may be a random copolymer, an alternating copolymer, a block copolymer, a graft polymer, or a copolymer containing two or more structures thereof. It is preferably a random copolymer from the aspects of the manufacturability of the polymer.

Polymer A1 is preferably a homopolymer of 2-(meth)acryloyloxyethylphosphorylcholine, more preferably a homopolymer of 2-methacryloyloxyethylphosphorylcholine.

While the weight average molecular weight of polymer A1 is not particularly limited, it is preferably 20,000 to 2,000,000, more preferably 100,000 to 1,500,000, further preferably 500,000 to 1,500,000. The weight average molecular weight can be calculated based on polyethylene glycol by gel permeation (hereinafter sometimes to be abbreviated as “GPC”).

For the formation of polymer A2, only one kind of 2-(meth)acryloyloxyethylphosphorylcholine (i.e., 2-acryloyloxyethylphosphorylcholine or 2-methacryloyloxyethylphosphorylcholine) may be used, or two kinds thereof (i.e., 2-acryloyloxyethylphosphorylcholine and 2-methacryloyloxyethylphosphorylcholine) may be used. As 2-(meth)acryloyloxyethylphosphorylcholine for the formation of polymer A2, 2-methacryloyloxyethylphosphorylcholine is preferred from the aspects of the preservation stability of the polymer and the availability of the starting material.

For the formation of polymer A2, only one kind of (meth)acrylic acid (i.e., acrylic acid or methacrylic acid) may be used, or, or two kinds thereof (i.e., acrylic acid and methacrylic acid) may be used. As (meth)acrylic acid, methacrylic acid is preferred from the aspects of the preservation stability of the polymer.

In the whole constitutional units of polymer A2, the proportion of a constitutional unit derived from (meth)acrylic acid is preferably 1 to 80 mol %, more preferably 30 to 80 mol %, further preferably 60 to 80 mol %. The rest of the constitutional unit of polymer A2 is preferably a constitutional unit derived from 2-(meth)acryloyloxyethylphosphorylcholine.

Polymer A2 may contain, as long as the effect of the present invention is not impaired, a constitutional unit derived from a monomer other than 2-(meth)acryloyloxyethylphosphorylcholine and (meth)acrylic acid (hereinafter to be indicated as “other monomer”). Examples of other monomer include alkyl(meth)acrylate (the carbon number of the alkyl group is preferably 1 to 6, more preferably 4 to 6), polyethylene glycol (meth)acrylate, benzyl (meth)acrylate, isobornyl (meth)acrylate, and the like.

In the whole constitutional units of polymer A2, the proportion of a constitutional unit derived from other monomer is preferably not more than 20 mol %, more preferably not more than 10 mol %, further preferably not more than 5 mol %. More preferably, polymer A2 does not contain a constitutional unit derived from other monomer.

Polymer A2 is preferably a copolymer of 2-(meth)acryloyloxyethylphosphorylcholine and (meth)acrylic acid, more preferably a copolymer of 2-methacryloyloxyethylphosphorylcholine and methacrylic acid.

While the weight average molecular weight of polymer A2 is not particularly limited, it is preferably 10,000 to 1,000,000, more preferably 100,000 to 1,000,000, further preferably 500,000 to 1,000,000.

As each monomer for the preparation of the polymer of the present invention, a commercially available product may be used, or the monomer may be produced by a known method. The polymer of the present invention can be produced by known methods (e.g., the method described in WO 2018/216628).

### [Other Component]

The reaction composition of the present invention may contain other components besides water, an amplification target nucleic acid, and the polymer of the present invention. Examples of other component include buffer, substrate, primer, enzyme, fluorescence DNA staining reagent, fluorescent probe, passive reference, nucleic acid other than the an amplification target nucleic acid, and the like. Only one kind of other component may be used, or two or more kinds thereof may be used in combination.

The aforementioned buffer is not particularly limited. Examples thereof include buffers with a pH adjusted to 6 to 9, more preferably about 7 to 8, by mixing a base such as tris(hydroxymethyl)aminomethane, Tricine, Bicine, and the like and an acid such as sulfuric acid, hydrochloric acid, acetic acid, phosphoric acid, and the like. The buffer desirably contains a magnesium salt and/or a manganese salt as appropriate. The buffer may further contain a salt such as potassium chloride, ammonium sulfate, and the like. The buffer may further contain a water-soluble organic solvent such as dimethyl sulfoxide, dimethylformamide, formamide, glycerol, and the like. The buffer may further contain a surfactant such as polyoxysorbitan fatty acid ester, polyoxyethylene alkylphenyl ether, and the like. The buffer may further contain a protein such as bovine serum albumin and the like. The buffer may further contain a water-soluble polymer such as polyethylene glycol and the like.

While the aforementioned substrate is not particularly limited, examples thereof include a mixture (dNTPs) of deoxyadenosine triphosphate (dATP), deoxythymidine triphosphate (dTTP), deoxyguanosine triphosphate (dGTP), and deoxycytidine triphosphate (dCTP). It is also possible to partially and/or entirely substitute dTTP with deoxyuridine triphosphate (dUTP). In sequencing PCR and the like, an appropriate amount of a mixture of dideoxyadenosine triphosphate (ddATP), dideoxythymidine triphosphate (ddTTP), dideoxyguanosine triphosphate (ddGTP), and dideoxycytidine triphosphate (ddCTP), or a fluorescently labeled product thereof may also be added.

The aforementioned primer is not particularly limited. Examples thereof include oligonucleotides with a length of about 15 to 30 bases designed and prepared by known methods. The primers may be subjected to, as appropriate, fluorescent labeling with fluorescein or the like, or isotope labeling with a heavy element, or the like.

The aforementioned enzyme is appropriately selected from DNA polymerase, restriction endonuclease, and the like, according to the nucleic acid amplification method to be employed, but DNA polymerase is preferred. As the DNA polymerase, known DNA polymerase can be used. From the aspect of heat resistance, enzymes derived from thermophilic bacteria, thermophilic archaea, hyperthermophile, or hyperthermophile archaea, and mutant enzymes thereof are preferred. One more DNA polymerases are appropriately selected from a DNA-dependent DNA polymerase, an RNA-dependent DNA polymerase (reverse transcriptase), or an enzyme having both functions, depending on the kind of amplification target nucleic acid.

The aforementioned fluorescent DNA staining reagent is not particularly limited, and examples thereof include SYBR™ Green I and the like.

The aforementioned fluorescent probe is not particularly limited, and examples thereof include TagMan™ probe.

The aforementioned passive reference may be selected appropriately according to the purpose of nucleic acid amplification. Examples of the passive reference include 50× ROX Passive Reference (manufactured by NIPPON GENE CO., LTD.) and the like.

Examples of the nucleic acid other than an amplification target nucleic acid include DNA and RNA as exogenous control genes, and the like. The aforementioned nucleic acid may be synthesized in vitro, or may be prepared from cells, microorganisms, viruses, or the like by known methods. Here, the cells, microorganisms, viruses, and the like may be those collected from human, animals, or plants in the natural world or environment, or may be those obtained by isolation and culture.

The reaction composition of the present invention may further contain an oil such as mineral oil or the like. In addition, the reaction composition of the present invention may further contain a solid support such as silica bead, magnetic bead, or the like.

It is also preferably performed to select a plurality of each component mentioned above and mix them in advance to give a master-mix (sometimes called primer mix, pre-mix, etc.).

### [Nucleic Acid Amplification Method]

The present invention also provides a nucleic acid amplification method using the reaction composition of the present invention (more specifically, a nucleic acid amplification method including performing a nucleic acid amplification reaction using the reaction composition of the present invention). Conditions and apparatuses for nucleic acid amplification methods (particularly PCR method) are well known, and those skilled in the art can appropriately perform nucleic acid amplification methods. For example, the reaction composition of the present invention is prepared in a container such as a 0.1 mL plastic tube and kept warm according to a temperature program for the process of the nucleic acid amplification method, whereby the desired sequence in the amplification target nucleic acid in the reaction composition of the present invention is preferably amplified.

The nucleic acid amplification method of the present invention is preferably a PCR method. When the amplification target nucleic acid is DNA, the nucleic acid amplification method of the invention is preferably a typical PCR method. When the amplification target nucleic acid is RNA, the nucleic acid amplification method of the present invention is preferably an RT-PCR method.

### [Method for Improving Repeatability of Nucleic Acid Amplification Method]

The present invention also provides a method for improving the repeatability of nucleic acid amplification methods, which includes performing a nucleic acid amplification reaction using the reaction composition of the present invention. Conditions and apparatuses for nucleic acid amplification reactions (particularly PCR reaction) are well known, and those skilled in the art can appropriately carry out nucleic acid amplification reactions.

The nucleic acid amplification reaction performed in the improvement method of the present invention is preferably a PCR reaction. When the amplification target nucleic acid is DNA, the nucleic acid amplification reaction is preferably a typical PCR reaction. When the amplification target nucleic acid is RNA, the nucleic acid amplification reaction is preferably an RT-PCR reaction.

### [Test Method]

The present invention also provides a genetic testing method using the nucleic acid amplification method of the present invention or the improvement method of the present invention (sometimes to be abbreviated as the “test method of the present invention” in the present specification). Specifically, the test method of the present invention is a method for examining the aforementioned nucleic acid (i.e., gene), including performing a nucleic acid amplification reaction using the reaction composition of the present invention containing water, an amplification target nucleic acid that may be contained in a sample at a concentration of not more than 10,000 copies/mL, and one or more polymers selected from the following group A at a concentration of 0.005 to 0.5 w/v % to improve repeatability of the nucleic acid amplification method. The aforementioned reaction composition can be prepared by mixing water, a sample, and the aforementioned polymer. When a sample itself contains water, the aforementioned reaction composition can be prepared by mixing the sample and the polymer.

Examples of the aforementioned test include medical tests, veterinary tests, forensic tests, medicine tests, food inspections, environmental audits, and the like. The test method of the present invention is preferably used for medical tests.

In the test method of the present invention, the determination of the presence or absence of the test subject based on the results of the nucleic acid amplification method of the present invention or the improvement method of the present invention can be performed using known methods. Examples of the method include
(1) a method including purifying an amplification reaction product and measuring the turbidity in the ultraviolet region of the purified nucleic acid solution,
(2) a method including developing an amplification reaction product by gel electrophoresis, staining the nucleic acid with a nucleic acid staining reagent such as ethidium bromide and the like, and measuring the staining intensity of the target band by visual observation or by instrumental analysis,
(3) a method including adding a fluorescent reagent such as CYBR™ Green and the like to a reaction composition of the nucleic acid amplification reaction and monitoring an increase in fluorescence intensity as the nucleic acid amplification reaction progresses, and the like.

The components other than the amplification target nucleic acid of the reaction composition of the present invention are mixed in advance, filled in a given reaction container which is combined with a member such as a sample collection instrument to prepare a test kit. Genetic tests can be performed by mixing the test kit with a sample containing an amplification target nucleic acid. The aforementioned test kit is preferably a pharmaceutical product for in vitro diagnosis.

### EXAMPLES

The present invention is explained in more detail in the following by referring to Examples and the like, which are not to be construed as limitative.

### Synthesis of polymer

### Synthetic Example 1

### Polymer A1 corresponds to polymer a1 was prepared as follows

160 g of 2-methacryloyloxyethylphosphorylcholine (hereinafter indicated as “MPC”) was weighed into a glass flask for polymerization, 260 g of purified water was added to dissolve MPC, and 3.7 g of tert-butyl hydroperoxide (hereinafter indicated as “BHP”) was added as a polymerization initiator to the obtained solution. Polymerization was performed by heating at 70° C. for 6 hr while stirring. The obtained reaction solution was ice-cooled and added dropwise to acetone for purification by reprecipitation. The obtained precipitate was collected by filtration, and vacuum dried to isolate a solid polymer a1 as a white powder. The weight average molecular weight of polymer a1 was 1,010,000 based on polyethylene glycol as measured by GPC under the below-mentioned conditions.

### Synthetic Example 2

### Polymer a2 corresponding to polymer A2 was prepared as follows

MPC (40 g) and methacrylic acid (hereinafter indicated as “MAc”) (27 g) (MPC/MAc=30/70 (molar ratio)) were weighed into a glass flask for polymerization, 391 g of purified water was added to dissolve them. BHP (3.3 g) was added to the obtained solution. Thereafter, in the same manner as in Synthetic Example 1, polymer a2 was obtained. The weight average molecular weight of polymer a2 was 730,000 based on polyethylene glycol as measured by GPC under the below-mentioned conditions.

### Synthetic Example 3

### Polymer b1 which is outside the range of the polymer of the present invention was prepared as follows

MPC (11.7 g) and stearyl methacrylate (hereinafter indicated as “SMA”) (3.3 g) (MPC/SMA=80/20 (molar ratio)) were weighed into a glass flask for polymerization, 85.0 g of ethanol were added to dissolve them, and AIBN (0.06 g) was added to the obtained solution. After the inside of the reaction container was sufficiently replaced with nitrogen, polymerization was performed by heating at 60° C. for 6 hr while stirring. Thereafter, in the same manner as in Synthetic Example 1, polymer b1 was obtained. The weight average molecular weight of polymer b1 was 43,000 based on polyethylene glycol as measured by GPC under the below-mentioned conditions.

The polymer obtained as described above was dissolved in Water, Nuclease free (manufactured by Nippon Gene Co., Ltd., hereinafter the same) such that the concentration was 10 times the final concentration described in the respective conditions of the below-mentioned Examples and Comparative Examples, and the obtained polymer aqueous solutions were used.

### [Gpc Measurement]

GPC measurement of each polymer obtained in Synthetic Examples 1 to 3 was performed under the following conditions.

GPC system: EcoSEC system (manufactured by Tosoh Corporation)

column: Shodex OHpak SB-802.5 HQ (manufactured by Showa Denko K. K.), and SB-806M HQ (manufactured by Showa Denko K.K.) connected in tandem
eluent: 20 mM sodium phosphate buffer (pH 7.4)
detector: differential refractive index detector
molecular weight standard: EasiVial PEG/PEO (manufactured by Agilent Technologies)
flow rate: 0.5 mL/min
column temperature: 40° C.
sample: obtained polymer diluted with eluent to final concentration of 0.1 wt %
injection volume: 100 μL

The monomers used in Synthetic Examples 1 to 3 and molar ratios thereof, and weight average molecular weights of the obtained polymers are shown in Table 1.

TABLE 1

| | monomer | monomer | molar ratio of | weight average |
|---|---|---|---|---|
| | 1 | 2 | monomers ^{(*1)} | molecular weight |
| polymer a1 | MPC | — | 100/0 | 1,010,000 |
| polymer a2 | MPC | MAc | 30/70 | 730,000 |
| polymer b1 | MPC | SMA | 80/20 | 43,000 |

^{(*1)} molar ratio of monomers = monomer 1/monomer 2

### Experimental Example 1

As the first series of Experimental Examples (Experimental Example 1-1 to Experimental Example 1-4), a nucleic acid amplification qualitative test based on the RT-PCR method was performed. As the amplification target nucleic acid, standard RNA attached to the SARS-CoV-2 RT-qPCR Detection Kit (manufactured by FUJIFILM Wako Pure Chemical Corporation) was diluted with Water, Nuclease free, and the obtained RNA aqueous solution was used.

### [Reaction Composition]

Using the reaction reagents, the polymers, and the amplification target nucleic acids, 10 kinds of reaction compositions shown in Table 2-1 and Table 2-2 were prepared. Example 1-1 in Experimental Example 1-2, and Example 1-2 in Experimental Example 1-3 were within the range of the reaction composition of the present invention.

TABLE 2-1

| | | Experimental | | | Experimental | | |
|---|---|---|---|---|---|---|---|
| | | Example 1-1 | | | Example 1-2 | | |
| | | control | control | control | control | Example | Comparative |
| | component | 1-1 | 1-2 | 1-3 | 2 | 1-1 | Example 1-1 |
| reaction | 5x Reaction Buffer^{(*2)} | 4 | | | μL | | |
| reagent | 2 mM dNTPs^{(*2)} | 4 | | | μL | | |
| | 50 mM Manganese(II) Acetate^{(*2)} | 1 | | | μL | | |
| | Hot Start Reverse Transcription | 0.25 | | | μL | | |
| | DNA Polymerase^{(*2)} | | | | | | |
| | primer 1-F^{(*3)} | 1 | | | μL | | |
| | primer 1-R^{(*4)} | 1.4 | | | μL | | |
| | probe 1-P^{(*5)} | 0.8 | | | μL | | |
| | 50 x ROX Passive Reference^{(*6)} | 0.4 | | | μL | | |
| | Water, Nuclease free | rest | | | | | |
| polymer ^{(*7)} | kind | — | polymer | polymer | — | polymer | polymer |
| | | | a1 | b1 | | a1 | b1 |
| | final concentration [w/v %] | — | 0.1 | 0.05 | — | 0.1 | 0.05 |

| nucleic | kind | — | Positive Control RNA, |
|---|---|---|---|
| acid ^{(*8)} | | | N set No. 2 (N2)^{(*2)} |

| | | | | |
|---|---|---|---|---|
| final concentration [copy/mL] | — | 2,500 | 2,500 | 2,500 |

| | | |
|---|---|---|
| total volume | 20 | μL |

TABLE 2-2

| | | Experimental | | Experimental | |
|---|---|---|---|---|---|
| | | Example 1-3 | | Example 1-4 | |
| | | control | Example | control | Comparative |
| | component | 3 | 1-2 | 4 | Example 1-2 |
| reaction | 5x Reaction Buffer^{(*2)} | 4 | | μL | |
| reagent | 2 mM dNTPs^{(*2)} | 4 | | μL | |
| | 50 mM Manganese (II) Acetate^{(*2)} | 1 | | μL | |
| | Hot Start Reverse Transcription | 0.25 | | μL | |
| | DNA Polymerase^{(*2)} | | | | |
| | primer 1-F^{(*3)} | 1 | | μL | |
| | primer 1-R^{(*4)} | 1.4 | | μL | |
| | probe 1-P^{(*5)} | 0.8 | | μL | |
| | 50 x ROX Passive Reference^{(*6)} | 0.4 | | μL | |
| | Water, Nuclease free | rest | | | |
| polymer ^{(*7)} | kind | — | polymer a1 | — | polymer a1 |
| | final concentration [w/v %] | — | 0.1 | — | 0.1 |
| nucleic | kind | Positive Control RNA, | | | |
| acid ^{(*8)} | | N set No. 2 (N2)^{(*2)} | | | |
| | final concentration [copy/mL] | 625 | 625 | 25,000 | 25,000 |

| | | |
|---|---|---|
| total volume | 20 | μL |

^{(*2)} component of SARS-CoV-2 RT-qPCR Detection Kit (manufactured by FUJIFILM Wako Pure Chemical Corporation)
^{(*3)} 5′-AAATTTTGGGGACCAGGAAC-3′ (SEQ ID NO: 1) (manufactured by eurofins) was dissolved in Water, Nuclease free, and the obtained 10 μM aqueous solution was used
^{(*4)} 5′-TGGCAGCTGTGTAGGTCAAC-3′ (SEQ ID NO: 2) (manufactured by eurofins) was dissolved in Water, Nuclease free, and the obtained 10 μM aqueous solution was used.
^{(*5)} 5′-[FAM]ATGTCGCGCATTGGCATGGA[BHQ1]-3′ (SEQ ID NO: 3) (manufactured by eurofins) was dissolved in Water, Nuclease free, and the obtained 5 μM aqueous solution was used.
^{(*6)} manufactured by NIPPON GENE CO., LTD., hereinafter the same
^{(*7)} 2 μL of a polymer aqueous solution with a concentration 10 times the final concentration was added. “—” means no addition.
^{(*8)} Amplification target nucleic acid. 5 μL of a nucleic acid aqueous solution with a concentration 4 times the final concentration was added. “—” means no addition.

In the above-mentioned Table 2 and the attached Sequence Listing, “FAM” means carboxyfluorescein and “BHQ1” means black hole quencher 1.

### [Nucleic Acid Amplification Reaction]

Each of the above-mentioned reaction compositions was prepared in a PCR plate (MicroAmp™ Fast Optical 96-well Reaction Plate with Barcode, 0.1 mL, manufactured by AppliedBioscience). This PCR plate was set on a StepOnePlus™ Real-Time PCR System (manufactured by AppliedBioscience), and a nucleic acid amplification reaction was performed using the temperature program shown in Table 3. The fluorescence intensity of probe 1-P-derived FAM, and the fluorescence intensity of passive reference (50× ROX Passive Reference) were read. The outputted ΔRn values are hereafter indicated as signal values. The signal value of the final cycle (i.e., 60 cycles) is indicated as an end-point value.

TABLE 3

| step # | temperature | time | | cycle number | reading |
|---|---|---|---|---|---|
| 1 | 90° C. | 30 | sec | | |
| 2 | 60° C. | 5 | min | | |
| 3 | 95° C. | 1 | min | | |
| 4 | 95° C. | 3 | sec | 60 cycles | |
| 5 | 60° C. | 20 | sec | | ∘ |

| | | |
|---|---|---|
| 6 | 20° C. | ∞ |

### [Results]

The results of Experimental Example 1-1 are shown in Table 4. These are the amplification results of the negative control not containing an amplification target nucleic acid, and the signal values obtained here can be said to be experimental non-specific signal values.

From the results of Experimental Example 1-1, “ΔRn=0.2”, which is 10 times the maximum value of ΔRn, was set as the threshold. In Experimental Example 1-2 to Experimental Example 1-4, an end-point value exceeding the aforementioned threshold was judged as “positive”, and other cases were judged “negative”. However, when a cycle number at the time when the signal value exceeded the threshold for the first time (hereinafter sometimes referred to as “C_{T} value”) exceeded 45, it was determined as “negative”.

TABLE 4

| | | Experimental Example 1-1 | | |
|---|---|---|---|---|
| | | control | control | control |
| | | 1-1 | 1-2 | 1-3 |
| polymer | kind | — | polymer a1 | polymer b1 |
| | final concentration | — | 0.1 | 0.05 |
| | [w/v %] | | | |
| nucleic | kind | — | | |
| acid ^{(*9)} | final concentration | — | | |
| | [copy/mL] | | | |
| results ^{(*10)} | #1 | 0.02 | 0.02 | 0.02 |
| | #2 | 0.02 | 0.02 | 0.02 |
| | #3 | 0.02 | 0.02 | 0.02 |
| | #4 | 0.02 | 0.02 | 0.02 |
| | average | 0.02 | 0.02 | 0.02 |

^{(*9)} amplification target nucleic acid
^{(*10)} maximum value of ΔRn
#1 to #4 show each result of 4 times (N = 4) of parallel tests.

The results of Experimental Example 1-2 to Experimental Example 1-4 are shown in Table 5-1 to Table 5-3. All of these are the amplification results of the positive control to which the amplification target nucleic acid was added, and all of the parallel tests become positive under ideal conditions. Repeatability was evaluated by comparing the number of positive judgments actually obtained.

In the results of Experimental Example 1-2, the number of positive judgments for control 2 was 3, whereas the number of positive judgments for Example 1-1 using polymer a1 was 9. Therefore, it is clear that the repeatability of the nucleic acid amplification method is markedly improved by using the reaction composition of the present invention.

The results of Experimental Example 1-3 are the amplification results when the concentration of the amplification target nucleic acid was set lower than that in Experimental Example 1-2, and it is clear that the repeatability of Example 1-2 using polymer a1 is high.

The results of Experimental Example 1-4 are amplification results when the concentration of the amplification target nucleic acid was set higher than that in Experimental Example 1-2. In this case, the number of positive judgments did not change depending on the presence or absence of the polymer of the present invention. Therefore, it is clear that the effects of the present invention are preferably exhibited when the concentration of the amplification target nucleic acid is extremely low.

TABLE 5-1

| | | | Experimental | | | Experimental | | Experimental | |
|---|---|---|---|---|---|---|---|---|---|
| | | | Example 1-2 | | | Example 1-3 | | Example 1-4 | |
| | | | control | Example | Comparative | control | Example | control | Comparative |
| | | | 2 | 1-1 | Example 1-1 | 3 | 1-2 | 4 | Example 1-2 |
| polymer | kind | | — | polymer | polymer | — | polymer | — | polymer |
| | | | | a1 | b1 | | a1 | | a1 |
| | final concentration [w/v %] | | — | 0.1 | 0.05 | — | 0.1 | — | 0.1 |
| nucleic | kind | | Positive Control RNA, N set No. 2 (N2) | | | | | | |
| acid ^{(*11)} | final concentration [copy/mL] | | 2,500 | 2,500 | 2,500 | 625 | 625 | 25,000 | 25,000 |
| results ^{(*12)} | #1 | end-point value | −0.07 | 2.39 | −0.03 | −0.06 | 2.67 | 2.94 | 3.63 |
| | | C_{T} value | nd | 38.0 | nd | nd | 38.4 | 34.0 | 34.0 |
| | | judgment | negative | positive | negative | negative | positive | positive | positive |
| | #2 | end-point value | −0.09 | 1.80 | 2.51 | −0.05 | 0.96 | 2.54 | 3.87 |
| | | C_{T} value | nd | 38.0 | 38.9 | nd | 42.2 | 33.0 | 33.8 |
| | | judgment | negative | positive | positive | negative | positive | positive | positive |
| | #3 | end-point value | −0.06 | 2.63 | 2.91 | −0.11 | 3.27 | 2.81 | 3.45 |
| | | C_{T} value | nd | 38.7 | 39.8 | nd | 40.7 | 33.8 | 34.4 |
| | | judgment | negative | positive | positive | negative | positive | positive | positive |

TABLE 5-2

| | | | Experimental | | | Experimental | | Experimental | |
|---|---|---|---|---|---|---|---|---|---|
| | | | Example 1-2 | | | Example 1-3 | | Example 1-4 | |
| | | | control | Example | Comparative | control | Example | control | Comparative |
| | | | 2 | 1-1 | Example 1-1 | 3 | 1-2 | 4 | Example 1-2 |
| results ^{(*12)} | #4 | end-point value | −0.04 | −0.04 | −0.03 | 0.06 | −0.08 | 3.99 | 3.51 |
| | | C_{T} value | nd | nd | nd | nd | nd | 33.9 | 34.4 |
| | | judgment | negative | negative | negative | negative | negative | positive | positive |
| | #5 | end-point value | 0.38 | 2.16 | −0.03 | −0.08 | 3.25 | 2.65 | 3.09 |
| | | C_{T} value | 40.9 | 38.4 | nd | nd | 37.6 | 33.9 | 35.0 |
| | | judgment | positive | positive | negative | negative | positive | positive | positive |
| | #6 | end-point value | 0.30 | 3.69 | 2.23 | 0.40 | 2.82 | 1.10 | 3.51 |
| | | C_{T} value | 39.7 | 34.0 | 38.7 | 38.5 | 38.9 | 33.7 | 33.8 |
| | | judgment | positive | positive | positive | positive | positive | positive | positive |
| | #7 | end-point value | 0.15 | 2.45 | −0.03 | 0.46 | 0.76 | 2.27 | 3.60 |
| | | C_{T} value | nd | 38.5 | nd | 38.4 | 42.1 | 33.7 | 34.9 |
| | | judgment | negative | positive | negative | positive | positive | positive | positive |
| | #8 | end-point value | −0.07 | 1.29 | −0.03 | −0.05 | 3.08 | 3.16 | 3.33 |
| | | C_{T} value | nd | 38.2 | nd | nd | 38.6 | 34.5 | 34.4 |
| | | judgment | negative | positive | negative | negative | positive | positive | positive |

TABLE 5-3

| | | | Experimental | | | Experimental | | Experimental | |
|---|---|---|---|---|---|---|---|---|---|
| | | | Example 1-2 | | | Example 1-3 | | Example 1-4 | |
| | | | control | Example | Comparative | control | Example | control | Comparative |
| | | | 2 | 1-1 | Example 1-1 | 3 | 1-2 | 4 | Example 1-2 |
| results ^{(*12)} | #9 | end-point value | −0.06 | 2.79 | −0.03 | −0.07 | 2.51 | 2.46 | 3.94 |
| | | C_{T} value | nd | 38.6 | nd | nd | 39.5 | 33.7 | 34.4 |
| | | judgment | negative | positive | negative | negative | positive | positive | positive |
| | #10 | end-point value | 1.08 | 0.72 | −0.05 | 0.54 | −0.07 | 3.95 | 3.11 |
| | | C_{T} value | 39.2 | 39.2 | nd | 37.9 | nd | 33.9 | 35.2 |
| | | judgment | positive | positive | negative | positive | negative | positive | positive |

| | | | | | | | |
|---|---|---|---|---|---|---|---|
| number of | 3 | 9 | 3 | 3 | 8 | 10 | 10 |
| positive judgment | | | | | | | |

11: amplification target nucleic acid
12: #1 to #10 show each result of 10 times (N=10) of parallel tests.
nd in the column of C_{T} value means not determined

### Experimental Example 2

As the second Experimental Example, a nucleic acid amplification reaction system different from that of Experimental Example 1 was set, and repeatability was evaluated in the same manner as in Experimental Example 1.

### [Reaction Composition]

Using the reaction reagents, the polymers, and the amplification target nucleic acids, 6 kinds of reaction compositions shown in Table 6 were prepared. Example 2-1 in Experimental Example 2-2, and Example 2-2 in Experimental Example 2-3 were within the range of the reaction composition of the present invention.

TABLE 6

| | | Experimental | | Experimental | | Experimental | |
|---|---|---|---|---|---|---|---|
| | | Example 2-1 | | Example 2-2 | | Example 2-3 | |
| | | control | control | control | Example | control | Example |
| | component | 5-1 | 5-2 | 6 | 2-1 | 7 | 2-2 |
| reaction | Reaction Buffer and NTPs^{(*13)} | 8 | | | μL | | |
| reagent | Manganese(II) Acetate^{(*13)} | 1 | | | μL | | |
| | Primers and Probes No. 2^{(*13)} | 1 | | | μL | | |
| | Hot Start Reverse Transcription | 0.25 | | | μL | | |
| | DNA Polymerase^{(*13)} | | | | | | |
| | 50x ROX | 0.4 | | | μL | | |
| | Water, Nuclease free | rest | | | | | |
| polymer ^{(*14)} | kind | — | polymer | — | polymer | — | polymer |
| | | | a2 | | a2 | | a2 |
| | final concentration [w/v %] | — | 0.05 | — | 0.05 | — | 0.05 |

| nucleic | kind | — | Positive Control RNA, N gene^{(*13)} | | | |
|---|---|---|---|---|---|---|
| acid ^{(*15)} | final concentration [copy/mL] | — | 2,500 | 2,500 | 1,250 | 1,250 |

| | | |
|---|---|---|
| total volume | 20 | μL |

^{(*13)}components of SARS-CoV-2 RT-qPCR Detection Kit Ver. 2 (manufactured by FUJIFILM Wako Pure Chemical Corporation)
^{(*14)} 2 μL of a polymer aqueous solution with a concentration 10 times the final concentration was added. “—” means no addition.
^{(*15)} Amplification target nucleic acid. 5 μL of a nucleic acid aqueous solution with a concentration 4 times the final concentration was added. “—” means no addition.

### [Nucleic Acid Amplification Reaction]

Each of the above-mentioned reaction compositions was prepared in a PCR plate (MicroAmp™ Fast Optical 96-well Reaction Plate with Barcode, 0.1 mL, manufactured by AppliedBioscience). This PCR plate was set on a StepOnePlus™ Real-Time PCR System (manufactured by AppliedBioscience), and a nucleic acid amplification reaction was performed using the temperature program shown in Table 7. The fluorescence intensity of FAM derived from TaqMan™ probe contained in Primers and Probes No. 2, and the fluorescence intensity of passive reference (50× ROX Passive Reference) were read. The outputted ΔRn values are hereafter indicated as signal values. The signal value of the final cycle (i.e., 60 cycles) is indicated as an end-point value.

TABLE 7

| step # | temperature | time | | cycle number | reading |
|---|---|---|---|---|---|
| 1 | 90° C. | 30 | sec | | |
| 2 | 60° C. | 10 | min | | |
| 3 | 95° C. | 1 | min | | |
| 4 | 95° C. | 3 | sec | 60 cycles | |
| 5 | 60° C. | 20 | sec | | ∘ |

| | | |
|---|---|---|
| 6 | 20° C. | ∞ |

### [Results]

The results of Experimental Example 2-1 are shown in Table 8. These are the amplification results of the negative control not containing an amplification target nucleic acid, and the signal values obtained here can be said to be experimental non-specific signal values.

From the results of Experimental Example 2-1, “ΔRn=0.2”, which is 10 times the maximum value of ΔRn, was set as the threshold. In Experimental Example 2-2 to Experimental Example 2-3, an end-point value exceeding the aforementioned threshold was judged as “positive”, and other cases were judged “negative”. However, when a cycle number at the time when the signal value exceeded the threshold for the first time (C_{T} value) exceeded 45, it was determined as “negative”.

TABLE 8

| | | Experimental Example 2-1 | |
|---|---|---|---|
| | | control 5-1 | control 5-2 |
| polymer | kind | — | polymer a2 |
| | final concentration [w/v %] | — | 0.05 |
| nucleic | kind | — | |
| acid ^{(*16)} | final concentration [copy/mL] | — | |
| results ^{(*17)} | #1 | 0.02 | 0.02 |
| | #2 | 0.02 | 0.02 |
| | #3 | 0.02 | 0.02 |
| | #4 | 0.02 | 0.02 |
| | average | 0.02 | 0.02 |

^{(*16)} amplification target nucleic acid
^{(*17)} maximum value of ΔRn
#1 to #4 show each result of 4 times (N = 4) of parallel tests.

The results of Experimental Example 2-2 and Experimental Example 2-3 are shown in Table 9-1 and Table 9-2. All of these are the amplification results of the positive control to which the amplification target nucleic acid was added, and all of the parallel tests become positive under ideal conditions. Repeatability was evaluated by comparing the number of positive judgments actually obtained.

From the results of Experimental Examples 2-2 and 2-3, it is clear that the repeatability of the nucleic acid amplification method was favorably improved in Examples 2-1 and 2-2 using the polymer a2.

TABLE 9-1

| | | | Experimental | | Experimental | |
|---|---|---|---|---|---|---|
| | | | Example 2-2 | | Example 2-3 | |
| | | | control | Example | control | Example |
| | | | 6 | 2-1 | 7 | 2-2 |
| polymer | kind | | — | polymer | — | polymer |
| | | | | a2 | | a2 |
| | final concentration [w/v %] | | — | 0.05 | — | 0.05 |
| nucleic | kind | | Positive Control RNA, N gene | | | |
| acid ^{(*18)} | final concentration [copy/mL] | | 2,500 | 2,500 | 1,250 | 1,250 |
| results ^{(*19)} | #1 | end-point value | 0.19 | 0.89 | 0.13 | 1.23 |
| | | C_{T} value | nd | 41.5 | nd | 39.6 |
| | | judgment | negative | positive | negative | positive |
| | #2 | end-point value | 0.01 | 1.26 | 0.16 | −0.03 |
| | | C_{T} value | nd | 39.3 | nd | nd |
| | | judgment | negative | positive | negative | negative |
| | #3 | end-point value | 0.07 | 0.94 | −0.02 | 1.37 |
| | | C_{T} value | nd | 41.5 | nd | 40.7 |
| | | judgment | negative | positive | negative | positive |
| | #4 | end-point value | 0.16 | 1.03 | −0.02 | −0.03 |
| | | C_{T} value | nd | 39.6 | nd | nd |
| | | judgment | negative | positive | negative | negative |
| | #5 | end-point value | 0.27 | 1.45 | 0.06 | −0.03 |
| | | C_{T} value | 41.3 | 39 | nd | nd |
| | | judgment | positive | positive | negative | negative |
| | #6 | end-point value | 0.13 | 0.9 | 0.01 | 1.36 |
| | | C_{T} value | nd | 38.7 | nd | 40.8 |
| | | judgment | negative | positive | negative | positive |

TABLE 9-2

| | | | Experimental | | Experimental | |
|---|---|---|---|---|---|---|
| | | | Example 2-2 | | Example 2-3 | |
| | | | control | Example | control | Example |
| | | | 6 | 2-1 | 7 | 2-2 |
| results ^{(*19)} | #7 | end-point value | −0.02 | 0.82 | −0.02 | −0.03 |
| | | C_{T} value | nd | 39.7 | nd | nd |
| | | judgment | negative | positive | negative | negative |
| | #8 | end-point value | 0.14 | 1.23 | 0.21 | −0.03 |
| | | C_{T} value | nd | 39.1 | 55.7 | nd |
| | | judgment | negative | positive | negative | negative |
| | #9 | end-point value | 0.32 | −0.03 | 0.08 | 1.03 |
| | | C_{T} value | 40.6 | nd | nd | 40.7 |
| | | judgment | positive | negative | negative | positive |
| | #10 | end-point value | 0.26 | 0.48 | 0.08 | −0.03 |
| | | C_{T} value | 40.3 | 41.3 | nd | nd |
| | | judgment | positive | positive | negative | negative |

| | | | | |
|---|---|---|---|---|
| number of | 3 | 9 | 0 | 4 |
| positive judgment | | | | |

18: amplification target nucleic acid
19: #1 to #10 show each result of 10 times (N=10) of parallel tests.
nd in the column of C_{T} value means not determined

### Industrial Applicability

Using the reaction composition for nucleic acid amplification of the present invention, the repeatability of the nucleic acid amplification method can be enhanced. Therefore, the reaction composition for nucleic acid amplification of the present invention can be used to improve the reliability of nucleic acid amplification qualitative tests and improve the accuracy of nucleic acid quantification by the dPCR method.

This application is based on a patent application No. 2022-159813 filed in Japan, the contents of which are incorporated in full herein.

What is claimed is:

1. A method for improving repeatability of a nucleic acid amplification method, comprising performing a nucleic acid amplification reaction using a reaction composition comprising water, an amplification target nucleic acid at a concentration of not more than 10,000 copies/mL, and one or more polymers selected from the following group A at a concentration of 0.005 to 0.5 w/v %:
[group A]
(A1) a polymer consisting of a constitutional unit derived from 2-(meth)acryloyloxyethylphosphorylcholine
(A2) a copolymer containing a constitutional unit derived from 2-(meth)acryloyloxyethylphosphorylcholine and a constitutional unit derived from (meth)acrylic acid.

2. A genetic testing method using the method according to claim 1.
